(19) United States
(12) Patent Application Publication
(10) Pub. No.: US 2011/0003005 A1
(43) Pub. Date: Jan. 6, 2011

(54) Methods of Treating PDNV and PONV with Extended Release Ondansetron Compositions

(76) Inventors: Gopi Venkatesh, Vandalia, OH (US); Stephen Perrett, Princeton, NJ (US); Ruth Thieroff-Ekerdt, Mendham, NJ (US)

Correspondence Address: Cooley LLP, ATTN: Patent Group, 777 6th Street, N.W., Suite 1100, Washington, DC, 20001, US

(21) Appl. No.: 12/688,493

(22) Filed: Jan. 15, 2010

Related U.S. Application Data
(60) Provisional application No. 61/223,218, filed on Jul. 6, 2009.

(51) Int. Cl. A61K 31/4178; A61K 9/50; A61K 9/58; A61P 41/00; A61P 1/08

(52) U.S. Cl. 424/494; 514/397; 424/497

(57) ABSTRACT

Extended release ondansetron compositions of the present invention are useful for treating postoperative nausea and vomiting (PONV) and/or postdischarge nausea and vomiting (PDNV).

### CROSS-REFERENCE TO RELATED APPLICATIONS

This application claims the benefit of U.S. Provisional Application No. 61/223,218, filed Jul. 6, 2009, which is herein incorporated by reference in its entirety for all purposes.

### BACKGROUND OF THE INVENTION

Postoperative nausea and vomiting after surgery (PONV) and postdischarge nausea and vomiting (PDNV) after ambulatory surgery are common post-surgical complications. PONV and PDNV are each recognized as separate clinical indications (see for example, Tong et al., “Consensus Guidelines for Managing Postoperative Nausea and Vomiting”, Anest. Analg. 2003, 97, pp. 62-71; Pan et al., “Antiemetic Prophylaxis for Postdischarge Nausea and Vomiting and Impact on Functional Quality of Living during Recovery in Patients with High Emetic Risks: A Prospective, Randomized, Double-Blind Comparison of Two Prophylactic Antiemetic Regimens”, Ambulatory Anesthesiology, vol. 107, No. 2, pp. 429-438, August 2008). For example, the risk of nausea and vomiting immediately post-surgery (PONV) has been shown to be different from the risk of nausea and vomiting following discharge (PDNV), and the risk factors for PONV and PDNV are different. In addition, the effectiveness of conventional antiemetic therapies in the period immediately post-surgery (PONV) versus the period after discharge (PDNV) has been shown to be different. The overall incidence of PONV following inpatient surgery is estimated to be around 20 to 30%. PONV is not caused by a single event, and is a common complaint, post-operatively occurring within the first 2 hr period in up to 80% of patients.

PONV typically refers to nausea and vomiting which occurs after surgery, such as immediately after surgery. PDNV refers to post-surgical nausea and vomiting, but specifically refers to the nausea and vomiting occurring after the patient has been discharged, after the immediate effects of anesthesia have worn off and the patient is relatively ambulatory. In addition, PDNV occurs outside of the hospital setting, such that nausea and vomiting are less readily controlled, in settings where conventional intravenous antiemetic therapies are not readily available.

The chemical triggering zone (CTZ) for nausea and vomiting is located at the area postrema on the floor of the 4^{th} ventricle of the brain, and raised intracerebral pressure is thought to cause vomiting via increased pressure at the ventricle. The CTZ is extremely sensitive to emetic stimuli. Various neurotransmitter types and receptors have been implicated in nausea and vomiting, including serotonin, acetylcholine, dopamine, muscarine, neurokinin-1, histamine, opioid, and 5-HT₃. Stimulation of the vestibular-cochlear, glossopharyngeal, or vagus nerves may also be involved. Accordingly, the risk factors for PONV and PDNV are complex, and known antiemetic agents vary widely in their effectiveness for treating PONV and PDNV.

Antiemetics are typically administered intravenously during surgery (e.g., in the final stages of surgery) in order to have an immediate prophylactic effect, and are often not administered subsequently unless or until the patient experiences nausea and/or vomiting. In some cases, oral, immediate release antiemetics are administered. Oral dosage forms differ from intravenous dosage forms in that the oral dosage form often has lower bioavailability, e.g., due to first-pass metabolism.

PONV and PDNV can result in patient discomfort (mild to severe), but can also have significant clinical consequences such as resulting in damage to delicate surgical sites, prolonging the time patients stay in post anesthesia care units, interrupting or delaying the administration of oral medications or fluid/food intake, and ultimately cause unplanned readmission or hospitalization following ambulatory surgery, thereby increasing medical costs (Kovac, A L. Drugs; 59(2): 213-243).

About 30% of those patients at moderate to high risk of PDNV following outpatient surgery (e.g., gynecological ambulatory surgery) experience nausea and vomiting after discharge, and about 36% of the patients who ultimately experience PDNV do not experience any nausea or vomiting prior to discharge (Anesthesiology 2002; 96: 994-1003), and thus are unlikely to be treated with antiemetics prior to discharge. Although PDNV events have not been scrutinized to the same extent as PONV, some patients will experience PDNV for up to 5 days (Caroll N V, et al. Ansesth. Analg. 1995; 80(5):903-909; Pfisterer M, et al. Ambul Surg. 2001; 9(1): 13-18; Odom-Forren J and Moser D K. J. Ambul. Surg. 2005; 12: 99-105).

5-HT₃ receptor antagonists such as ondansetron are highly specific and selective for nausea and vomiting, and are known to be most effective when given orally prior to surgery, intravenously (IV) at the end of surgery, or IV after surgery in the early part (i.e., 0-2 hr period) of PONV. The recommended IV dose of ondansetron is 4 to 8 mg IV in adults, and 50 to 100 μg/kg in children.

As a practical matter, it is difficult or inconvenient to administer IV antiemetics post-discharge. Oral administration is more convenient, less costly, and safer than IV administration. Accordingly, it would be advantageous to provide orally administered antiemetics effective to prevent PONV or PDNV in at least the first 24 hr period following surgery. However, the effectiveness of orally administered ondansetron for treating or preventing PONV or PDNV is at best equivocal. Some studies show that orally administered ondansetron is ineffective in improving control of nausea and vomiting in the 24 hour period following surgery (Kovac A L, O'Connor T A, Pearman M H, Kekoler L J, Edmondson D, Baughman V L, Angel J J, Campbell C, Jense H G, Mingus M L, Shahvari M B G, Creed M R. Efficacy of repeat intravenous dosing of ondansetron in controlling postoperative nausea and vomiting: a randomized, double-blind, placebo-controlled, multicenter trial. Journal of Clinical Anesthesia 1999; 11(6):453-459). Other studies that have evaluated the use of orally delivered ondansetron for periods longer than 24 hours post-discharge have given mixed results. See, for example, Thagaard et al., who found no difference between orally administered ondansetron and placebo over a 24 and 72 h period following surgery. (Thagaard K S, Steine S, Raeder J. Ondansetron disintegrating tablets of 8 mg twice a day for 3 days did not reduce the incidence of nausea and vomiting after laparoscopic surgery. Eur J Anaesth 2003; 20:153-157), whereas Gan et al. (Gan T J, Randall F, Reeves J, Ondansetron Orally Disintegrating Tablet Versus Placebo for the Prevention of Postdischarge Nausea and Vomiting After Ambulatory Surgery Anesth Analg 2002; 94:1199-1200) found that orally administered ondansetron did reduce nausea and vomiting post-discharge, relative to placebo. Still other studies have shown no effect for oral ondansetron administered in the 24 h period after surgery, but efficacy only for ondansetron administered after the 24 h period following surgery (Pan et al., “Antiemetic Prophylaxis for Postdischarge Nausea and Vomiting and Impact on Functional Quality of Living During Recovery in Patients with High Emetic Risks: A Prospective, Randomized, Double-Blind Comparison of Two Prophylactic Antiemetic Regimens”, Ambulatory Anesthesiology, vol. 107, No. 2, pp. 429-438, August 2008).

Ondansetron is currently available only as an immediate release tablet (conventional tablet or orally disintegrating tablet (ODT)). For immediate release dosage forms, the relatively short in-vivo half-life of ondansetron results in an ondansetron plasma concentration characterized by sharp peaks and troughs, thereby requiring that the dosage form be administered periodically in order to be effective over a 24-hour period. However, this type of pharmacokinetic profile is often associated with alternating periods of increased side effects and inefficacy as the plasma concentrations of drug cycle outside of the ideal therapeutic range. This cycling of drug plasma levels can result in the break through of symptoms, i.e. nausea and vomiting. This makes the therapeutic effect unpredictable both between patients and upon repeated dosing. Repeat dosing schedules also pose other problems for patients who are distressed, experiencing nausea and vomiting, and may have difficulty swallowing. To these factors are added the noncompliance with administration schedules associated with repeat dosage schedules. All of these factors reduce the effectiveness of prophylactic oral doses of antiemetics.

Thus, there is an unmet need for methods of treating PONV and/or PDNV with a once-daily antiemetic dosage form for patients at moderate to high risk of PONV/PDNV following inpatient or outpatient ambulatory surgery.

### SUMMARY OF THE INVENTION

The present invention is directed to a method of treating or preventing PONV or PDNV comprising orally administering to a surgical patient in need thereof, at least one extended release dosage form comprising a selective serotonin 5-HT₃ antagonist, prior to and/or after surgery.

In one embodiment, the extended release dosage form of the present method comprises TPR particles and IR particles; wherein the TPR particles each comprise a core coated with a TPR layer; the core comprises a selective serotonin 5-HT₃ antagonist and a pharmaceutically acceptable organic acid, wherein the selective serotonin 5-HT₃ antagonist and the pharmaceutically acceptable organic acid are separated from each other by an SR layer; the TPR layer comprises a water insoluble polymer and an enteric polymer; the SR layer comprises a water insoluble polymer; and the IR particles each comprise the selective serotonin 5-HT₃ antagonist, and release at least about 80 wt. % of the selective serotonin 5-HT₃ antagonist in about 5 minutes when dissolution tested using United States Pharmacopoeia dissolution methodology (Apparatus 2—paddles@ 50 RPM, 0.1N HCl at 37° C.

In a particular embodiment, the extended release dosage form of the present method comprises TPR particles and IR particles; wherein the TPR particles each comprise: an inert bead; an acid layer disposed over the inert bead, comprising the pharmaceutically acceptable organic acid such as fumaric acid; the SR layer disposed over the acid layer; a drug layer disposed over the SR layer (e.g., comprising ethyl cellulose, optionally plasticized), wherein the drug layer comprises a selective serotonin 5-HT₃ antagonist such as ondansetron (or a salt and/or solvate thereof); and the TPR layer (e.g., comprising ethyl cellulose and hydroxypropyl methylcellulose phthalate, optionally plasticized) is disposed over the drug layer. The IR particles comprise a granulate of the pharmaceutically acceptable organic acid (e.g. fumaric acid), the selective serotonin 5-HT₃ antagonist (e.g. ondansetron or a salt and/or solvate thereof), and an optional binder (e.g. hydroxypropyl cellulose), as well as one or more additional excipients (e.g. a fillers such as lactose and/or microcrystalline cellulose, a disintegrant such as crospovidone, etc.).

In most embodiments, the extended release dosage form is administered up to 5 times, once-daily, post discharge, for example in the morning following discharge, and once-daily up to about 4 additional times following the first dose.

### BRIEF DESCRIPTION OF THE DRAWINGS

FIG. 1.A illustrates the cross-section of an SR coated, fumaric acid-containing core.

FIG. 1.B illustrates a cross-section of a TPR bead comprising an SR coated, fumaric acid-containing core.

FIG. 2 illustrates the release profiles of both fumaric acid (“Acid”) and ondansetron hydrochloride (“Compound”) from the TSR beads of Example 1.

FIG. 3 illustrates the release profiles of ondansetron hydrochloride from the TSR beads of Example 2.

FIG. 4 illustrates the ondansetron plasma concentration—time profiles of MR capsule formulations (PF391EA0001, PF392EA0001, and PF379EA0001) comprising RR Granules (rapid release granules) and TPR beads of Example 3.

FIG. 5 illustrates the drug release profiles of the MR capsule formulations of Example 3 or 4 (pilot CTM: PF392EA0001, pivotal CTM: PF392EA0002, and pilot CTM: PF379EA0001).

FIG. 6A shows the plasma concentrations of ondansetron for 0-24 and 120-144 hours after administration of oral doses of 24 mg ondansetron MR dosage form and 8 mg Zofran® tablets administered twice daily.

FIG. 6B shows the plasma concentrations of ondansetron on day 1 after oral doses of 24 mg ondansetron MR dosage form and 8 mg Zofran® tablets administered twice daily.

FIG. 7A shows the plasma concentrations of ondansetron for 0-24 and 120-144 hours after administration of oral doses of 24 mg ondansetron MR dosage form and 8 mg Zofran® tablets administered thrice daily.

FIG. 7B shows the plasma concentrations of ondansetron on day 1 after oral doses of 24 mg ondansetron MR dosage form and 8 mg Zofran® tablets administered thrice daily.

FIG. 8A shows the plasma concentrations of ondansetron on day 1 after oral doses of 24 mg ondansetron MR dosage form and 8 mg Zofran® tablets administered once on day one.

FIG. 8B shows the plasma concentrations of ondansetron on day 3 after twice daily administration of oral doses of 24 mg ondansetron MR dosage foam and 8 mg Zofran® tablets (on day 3).

FIG. 8C shows the plasma concentrations of ondansetron on day 6 after trice daily administration of oral doses of 24 mg ondansetron MR dosage form and 8 mg Zofran® tablets (on day 6).

FIG. 9 shows a schematic representation of the model-based drug development approach.

FIG. 10A demonstrates the relationships between (0-2 hr) incidence rate (emesis or nausea rate) vs. (0-1 hr) exposure response for ondansetron.

FIG. 10B demonstrates the relationships between (0-2 hr) incidence rate (emesis or nausea rate) vs. (0-1 hr) exposure response for ondansetron with PONV history differences.

FIG. 10C demonstrates the relationships between (0-24 hr) incidence rate (emesis or nausea rate) vs. (0-1 hr) exposure response for ondansetron with PONV history differences.

FIG. 11A demonstrates the simulated relationships between (0-2 hr) or (0-24 hr) incidence rate (emesis or nausea rate) vs. (0-1 hr) exposure response for ondansetron.

FIG. 11B demonstrates the corresponding incidence exposure vs. (0-1 hr) exposure response.

FIG. 12A demonstrates the relationships between (0-2 hr) exposure incidence (emesis or nausea AUC) vs. (0-1 hr) exposure response for ondansetron with PONV history differences.

FIG. 12B demonstrates the relationships between (0-24 hr) exposure incidence (emesis or nausea AUC) vs. (0-1 hr) exposure response for ondansetron with PONV history differences.

FIG. 13A demonstrates the mean simulated relationships between (0-24 hr) incidence response (emesis or nausea) vs. exposure (AUC_{0-2 hr}) for ondansetron (line) with all data sets modeled.

FIG. 13B demonstrates the simulated relationship between (0-24 hr) methods using a once-daily 24 mg dose for post operative nausea or vomiting vs. Zofran® 8 mg (line) with 90% prediction intervals.

### DETAILED DESCRIPTION OF THE INVENTION

All documents cited herein are incorporated by reference in their entirety for all purposes; the citation of any document is not to be construed as an admission that it is prior art with respect to the present invention.

As used herein, various terms are defined as described in “How to study postoperative nausea and vomiting”, Acta Anaesthesiol. Scand. 2002:46:921-928:
“nausea” refers to a subjective sensation of an urge to vomit, in the absence of expulsive muscular movements; when severe, it is associated with increased salivary secretion, vasomotor disturbances, and sweating;
“vomiting” or “emesis” refers to the forcible expulsion through the mouth of the gastric contents. Vomiting results from coordinated activity of the abdominal, intercostals, laryngeal, and pharyngeal muscles;
“retching” refers to an unproductive effort to vomit, or the rhythmic action of respiratory muscles preceding vomiting;
“incidence” refers to a risk measure associated with developing some new condition within a specified period of time. Incidence=% patients with one or more events wherein an event is nausea, emesis or taking rescue medication;
“incidence rate” refers to the total number of incidence events divided by the duration of the observation interval in which the incidence events occurred, expressed as a rate (e.g., %/hour);
“exposure” refers to the area under the plasma concentration—time profile from time=0 to time=t (e.g., AUC_{0-2 hr}).

As used herein, as well as in specific examples thereof, reference to a drug or drug class (e.g., selective serotonin 5-HT₃ antagonist, ondansetron, etc.) includes the drug itself, as well as pharmaceutically acceptable salts, polymorphs, stereoisomers and mixtures thereof.

As used herein, the term “immediate release” (IR) refers to the release of greater than or equal to about 50%, in some embodiments greater than about 75%, or more than about 90%, and in certain embodiments greater than about 95% of the drug within about 30 minutes when dissolution tested in 0.1N HCl, or within about one hour following administration of the dosage form. Immediate release particles (IR particles) are drug-containing particles which provide immediate release of the drug.

As used herein, the term “rapid release” (RR) in regard to drug-containing particles, refers to drug-containing particles in which at least about 80% of the drug contained in particle is released in about 5 minutes, for example when dissolution tested using United States Pharmacopoeia (USP) dissolution methodology (Apparatus 2—paddles@ 50 RPM, 0.1N HCl at 37° C. For example, RR particles can include, but are not limited to particles in which the drug is layered on 45-60 mesh, or 60-80 mesh sugar spheres, as well as water-soluble microgranules comprising the drug and a filler, (e.g., lactose) and an organic acid (e.g., fumaric acid). Rapid release particles are a particular type of IR particles with relatively high rates of drug release.

The term “TPR (timed, pulsatile release) bead” or “TPR dosage form”, as defined here, is characterized by an immediate release pulse or a sustained release profile after a pre-determined lag time. The term “lag-time” refers to a time period wherein less than about 10%, more particularly substantially none, of the dose (drug) is released, and a lag-time of from at least about 2 to 10 hours is achieved by coating typically with a combination of water-insoluble and enteric polymers (e.g., ethylcellulose and hypromellose phthalate). Similarly, a TPR coating or TPR layer refers to a layer, membrane, or coating which provides such properties. As described herein, TPR coatings or layers comprise a pharmaceutically acceptable water insoluble polymer combined with an enteric polymer, optionally plasticized with one or more pharmaceutically acceptable plasticizers.

The term “SR layer”, “SR coating”, etc. refers to a layer or coating comprising a pharmaceutically acceptable water insoluble polymer, optionally plasticized with one or more pharmaceutically acceptable plasticizers.

The clinical terms “plasma concentration—time profile”, “Cₘₐₓ”, “AUC”, “Tₘₐₓ”, and “elimination half life” have their generally accepted meanings, and hence, are not redefined. Unless indicated otherwise, all percentages and ratios are calculated by weight based on the total composition.

The term “coating weight” refers to the dry weight of a coating as a percentage of the weight of the substrate prior to coating. For example, a 10 mg particle coated with 1 mg coating (dry weight) has a coating weight of 10%.

The present invention is a method of treating or preventing PONV and/or PDNV by orally administering a dosage form comprising a selective serotonin 5-HT₃ antagonist. The dosage form comprises TPR particles and IR particles (particularly RR particles), each comprising a selective serotonin 5-HT₃ antagonist (e.g. ondansetron). The TPR particles comprise a core comprising the selective serotonin 5-HT₃ antagonist and a pharmaceutically acceptable organic acid (e.g. fumaric acid) separated from each other by an SR layer comprising a water insoluble polymer (such as ethyl cellulose). The IR particles comprise the selective serotonin 5-HT₃ antagonist, and release at least 80 wt. % of the selective serotonin 5-HT₃ antagonist in about 5 minutes (using USP dissolution methodology (Apparatus 2—paddles @50 RPM in 0.1 N HCl at 37° C.)).

Oral dosage forms suitable for use in the method of the present invention provide extended release of the selective serotonin 5-HT₃ antagonist upon once-daily oral administration of the dosage form. The extended release dosage form can include diffusion systems (e.g. reservoir devices and matrix devices), dissolution systems (e.g., encapsulated dissolution systems such as “tiny time pills”), matrix dissolution systems, combination diffusion/dissolution systems, osmotic systems and ion-exchange resin systems as described in Remington's Pharmaceutical Sciences, 1990 ed., pp. 1682-1685.

In one embodiment, the oral dosage form for use in the method of the present invention can be prepared as described in co-pending U.S. patent application Ser. No. 12/209,285, filed Sep. 12, 2008 (which is herein incorporated by reference in its entirety for all purposes).

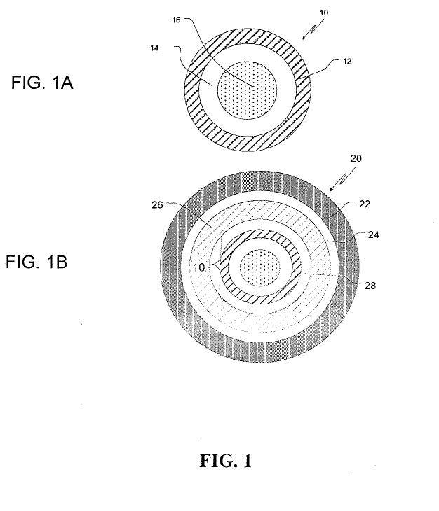

Specific embodiments of the present invention will be described in further detail with reference to the accompanying FIGS. 1.A and 1.B. In FIG. 1.A, an SR-coated core 10 comprising an SR coating 12 applied on an organic acid-containing core comprising a layer of a pharmaceutically acceptable organic acid in a binder 14 coated on an inert particle core 16. The inert particle core 16, organic acid-coating layer 14 and a dissolution rate controlling SR layer 12 make up the SR-coated organic acid-containing core 10. In FIG. 1.B, a representative TPR bead is illustrated. The TPR bead 20 comprises a lag-time coating 22 applied on a primary SR layer 24, a protective seal-coat 26 and a weakly basic drug layer 28 applied on an SR-coated acid-containing core 10. In certain embodiments of the present invention, the intermediate SR barrier layer is not applied, i.e., the TPR layer is directly applied over the seal coated immediate release beads.

In one embodiment, the pharmaceutical compositions suitable for use in the method of the present invention comprise a plurality of TPR and IR particles, wherein the TPR particles each comprise a core coated with a TPR layer; the core comprises a selective serotonin 5-HT₃ antagonist (e.g. ondansetron) and a pharmaceutically acceptable organic acid separated from each other by an SR layer; and the IR particles each comprise the selective serotonin 5-HT₃ antagonist (e.g. ondansetron) in combination with suitable excipients.

In a particular embodiment, the TPR particles comprise an inert core (e.g., a sugar bead etc.) sequentially coated with a pharmaceutically acceptable organic acid (e.g., fumaric acid) and a pharmaceutically acceptable binder (e.g., hydroxypropyl cellulose); a sustained release (SR) layer (e.g., comprising a pharmaceutically acceptable water insoluble polymer such as ethyl cellulose, optionally plasticized with a pharmaceutically acceptable plasticizer such as triethyl citrate or polyethylene glycol); a drug layer comprising the selective serotonin 5-HT₃ antagonist (e.g., ondansetron or a pharmaceutically acceptable salt and/or solvate thereof) and a pharmaceutically acceptable binder (e.g., povidone); an optional sealing layer (e.g. comprising a water soluble polymer such as hydroxypropyl methylcellulose); and a TPR layer (e.g., comprising a water insoluble polymer such as ethyl cellulose, an enteric polymer such as hydroxypropylmethylcellulose phthalate, and an optional pharmaceutically acceptable plasticizer such as triethyl citrate).

The IR particles release at least about 50% of the selective serotonin 5-HT₃ antagonist within about 30 minutes when dissolution tested in 0.1N HCl, or within about one hour following administration of the dosage form. In particular embodiments, the IR particles are RR particles, and release at least about 80 wt. % of the selective serotonin 5-HT₃ antagonist in about 5 minutes when dissolution tested using United States Pharmacopoeia (USP) dissolution methodology (Apparatus 2—paddles@ 50 RPM, 0.1N HCl at 37° C.

The RR particles can have any suitable structure which provides the required rapid release properties. For example, the RR particles can comprise the selective serotonin 5-HT₃ antagonist deposited on an inert core (e.g., sugar bead, optionally of smaller average diameter than the inert core of the TPR particles), optionally with a pharmaceutically acceptable binder. In other embodiments, the RR particles comprise the selective serotonin 5-HT₃ antagonist, granulated in the presence of a pharmaceutically acceptable polymeric binder, a pharmaceutically acceptable organic acid, and at least one excipient (e.g., one or more fillers such as lactose and/or microcrystalline cellulose; a disintegrant such as crospovidone, etc.).

In a particular embodiment, the extended release oral dosage form for use in the methods of the present invention comprises a capsule filled with a combination of TPR particles and RR particles, wherein the TPR particles comprise sugar beads sequentially coated with fumaric acid and a binder (e.g., hydroxypropyl cellulose); a sustained release (SR) layer comprising ethyl cellulose and an optional plasticizer (e.g., optionally triethyl citrate); a drug layer comprising ondansetron and a binder (e.g., povidone); an optional sealing layer (e.g. hydroxypropyl methylcellulose); and a TPR layer comprising ethyl cellulose, hydroxypropylmethylcellulose phthalate, and an optional plasticizer (e.g., optionally triethyl citrate); and the RR particles comprise a granulate of ondansetron, fumaric acid, crospovidone, microcrystalline cellulose, and hydroxypropyl cellulose.

A non-limiting list of selective serotonin 5-HT₃ antagonists suitable for use in the extended release compositions include ondansetron, tropisetron, granisetron, dolasetron, palonosetron, ramosetron, and salts and/or solvates thereof. In a particular embodiment, the selective serotonin 5-HT₃ antagonist is ondansetron, or salts and/or solvates thereof.

A non-limiting list of water-insoluble polymers, suitable for use in the TPR and SR layers includes ethylcellulose, cellulose acetate, polyvinyl acetate, neutral copolymers of ethyl acrylate and methylmethacrylate, copolymers of acrylic and methacrylic esters containing quaternary ammonium groups, and waxes. The water-insoluble polymer used in the TPR layer can be the same as or different from the water-insoluble polymer used in the SR layer. In a particular embodiment, the water-insoluble polymer for both the TPR and SR layers is ethylcellulose.

A non-limiting list of enteric polymers suitable for use in the TPR layer includes cellulose acetate phthalate, hydroxypropyl methylcellulose phthalate, hydroxypropyl methylcellulose acetate succinate, polyvinyl acetate phthalate, pH-sensitive copolymers of methacrylic acid and methylmethacrylate, and shellac. In a particular embodiment, the enteric polymer of the TPR layer is hydroxypropyl methylcellulose phthalate.

A non-limiting list of pharmaceutically acceptable organic acids includes citric acid, lactic acid, fumaric acid, malic acid, maleic acid, tartaric acid, succinic acid, oxalic acid, aspartic acid, and glutamic acid. In a particular embodiment, the pharmaceutically acceptable organic acid is fumaric acid.

As discussed herein, the TPR and SR layers can each optionally include a plasticizer. In some cases, it may be desirable to omit a plasticizer (e.g. in order to reduce cost, reduce exposure of patients to plasticizers, etc.). One of skill in the pharmaceutical arts can select suitable grades of water-insoluble polymers and/or enteric polymers amenable to forming a coating without plasticizer. Alternatively, it may be desirable to incorporate a plasticizer into one or both of the TPR and SR layers (e.g. in order to adjust the physical properties of the respective layers, or adjust the release rate of the drug and/or organic acid). When a plasticizer is used, a non-limiting list of suitable plasticizers includes triacetin, tributyl citrate, triethyl citrate, acetyl tri-n-butyl citrate, diethyl phthalate, dibutyl sebacate, polyethylene glycol, polypropylene glycol, castor oil, acetylated mono- and di-glycerides and mixtures thereof. When a plasticizer is used in both the TPR and SR layers, the plasticizer can be the same or different. In one embodiment, the plasticizer of the SR layer is triethyl citrate. In another embodiment, the plasticizer of the TPR layer is triethyl citrate. In yet another embodiment of plasticizer of both the TPR and SR layers is triethyl citrate.

As described herein, any type of oral extended release dosage form comprising a selective serotonin 5-HT₃ antagonist can be used in the method of the present invention. In one embodiment, the TPR particles comprise “layered beads” in which the organic acid and drug are layered onto an inert core. The inert core can be any pharmaceutically acceptable inert core; in particular those with an average particle size of 25-30 mesh. A non-limiting list of suitable inert cores includes sugar spheres, cellulose spheres, lactose spheres, lactose-MCC spheres, mannitol-MCC spheres, and silicone dioxide spheres.

Antiemetic drugs such as domperidone, granisetron, cyclizine, droperidol, dexamethasone, and ondansetron, as well as combinations of these drugs have been used to treat postoperative nausea and vomiting. Most commonly, antiemetic drugs are administered prophylactically by IV either prior to surgery, or immediately after surgery, and any breakthrough nausea and vomiting experienced postoperatively are treated with “rescue” doses of IV or immediate release oral antiemetics. Oral antiemetics are generally considered less effective than IV antiemetics because the “first pass” metabolism of oral antiemetics results in lower bioavailability. In addition, it may be difficult or impossible to administer an oral dosage form to patients suffering from postoperative nausea and vomiting.

Alternatively, the method of the present invention also includes administration of an oral extended release dosage form comprising a selective serotonin 5-HT₃ antagonist in combination with oral dosage forms comprising other types of antiemetic drugs. For example, the method of the present invention comprising treating or preventing PONV and/or PDNV by administering at least one extended release dosage form comprising a selective serotonin 5-HT₃ antagonist to a surgical patient in need thereof, in most embodiments after surgery or at discharge, and further administering at least one additional oral antiemetic comprising one or more NK-1 antagonist, dopamine antagonist, H1 histamine receptor antagonist, cannabinoid, benzodiazepine, anticholinergic, steroid, etc. The coadministration of the extended release dosage form comprising a selective serotonin 5-HT₃ antagonist in the additional oral antiemetic can include administration of the two dosage forms more or less simultaneously; or at different times, such that clinically significant plasma levels of the selective serotonin 5-HT₃ antagonist and the additional oral antiemetic are present in the patient.

In methods of the present invention in which an extended release dosage form comprising a selective serotonin 5-HT₃ antagonist is coadministered with an additional oral antiemetic, the NK-1 antagonist can include aprepitant or casopitant; the dopamine antagonist can include domperidone, droperidol, haloperidol, chlorpromazine, or prochlorperazine; the H1 histamine receptor antagonist can include cyclizine, diphenhydramine, dimenhydrinate, meclizine, promethazine, or hydroxyzine; the cannabinoid can include cannabis, dronabinol, or nabilone; the benzodiazepine can include midazolam or lorazepam; the anticholinergic can be scopalamine; and the steroid can be dexamethasone.

In the method of the present invention, the extended release oral dosage form can be administered prior to surgery, immediately after surgery, or at discharge, or can be used in combination with prophylactic administration of an IV antiemetic administered before, during, immediately after surgery, or at discharge. For example, the extended release dosage form can be administered prior to surgery instead of the prophylactic IV antiemetic, thereby providing an effective prophylactic dose of selective serotonin 5-HT₃ antagonist which provides protection against PONV/PDNV immediately after surgery, at discharge, as well as for an extended postoperative period. Alternatively, the IV antiemetic can be administered immediately before or following surgery, and the extended release dosage form comprising a selective serotonin 5-HT₃ antagonist can be administered prior to or at discharge, thereby providing effective protection against PONV and/or PDNV for an extended period of time, e.g. until the day following surgery. In either situation (either in combination with IV antiemetics or administered instead of IV antiemetics) the extended release dosage form comprising a selective serotonin 5-HT₃ antagonist can be further administered once-daily for one or more days following surgery in order to provide extended protection against PDNV. When administered after surgery, the extended release dosage form comprising a selective serotonin 5-HT₃ antagonist can be administered at discharge, and/or the day following discharge (e.g. in the morning following discharge), and optionally up to five days following discharge (e.g. once-a-day administration each morning following discharge).

The method of the present invention, e.g. as described in the examples, provides clinically significant prophylaxis, treatment, or amelioration of PONV and/or PDNV, which is equivalent or superior to the prophylaxis, treatment, or amelioration of PONV and/or PDNV provided by conventional therapies. In addition, as described herein the method of the present invention, employing a once-per-day extended oral release dosage form, is more convenient and more effective compared to conventional immediate release oral dosage forms, and safer than IV administration. The method of the present invention prevents the incidence of nausea and/or vomiting for at least 3 days following surgery, avoids unanticipated extensions of hospital stay (PONV) or IV administration of ondansetron before discharge (PDNV), provide enhanced patient compliance and quality of life, and also reduce medical costs.

The method of the present invention, as described herein, can be used for both inpatient and outpatient surgical procedures. For example, although intravenous administration is more readily available for inpatient procedures, the present method 5-HT₃ antagonist avoids the risks and expense associated with intravenous administration. For outpatient surgical procedures, it is generally difficult to administer antiemetics intravenously after discharge, and accordingly administration of an oral dosage faun is substantially more convenient and less costly. In addition, the present method of administering an extended release dosage form comprising a selective serotonin 5-HT₃ antagonist is a substantial improvement over the currently available immediate release dosage forms, because immediate release dosage forms require multiple daily administrations in order to provide continuous treatment or prophylaxis of PDNV, whereas the present method provides for once-daily administration, resulting in improved compliance and reduced incidence of PDNV. Thus, for example, the extended release dosage form comprising a selective serotonin 5-HT₃ antagonist described herein can be administered immediately prior to discharge and/or once-daily subsequent to discharge (e.g., beginning about 24 hours after discharge, for example in the morning following discharge) for up to one week (for example up to 5 days after discharge) to treat or ameliorate PONV and/or PDNV.

The method of the present invention can be used generally for both inpatient and outpatient surgery, or targeted to specific patients having a moderate or high level of risk factors for PONV or patients having a moderate or high level of risk factors for PDNV. For example, risk factors for PONY include being female, having a history of post-operative nausea and vomiting and/or having a history of motion sickness, nonsmoking status, and the administration of post-operative opioids. Risk factors for PDNV are somewhat different from those of PONV, and include being of young age, being female, having a history of post-operative nausea and vomiting, and/or a history of post-operative nausea and vomiting in a post-anesthesia care unit (PACU), and administration of perioperative opioids (including oral opioid analgesics). Accordingly, in one embodiment, the present invention includes administration of an extended release dosage form comprising a selective serotonin 5-HT₃ antagonist, prior to discharge, to a patient having one or more of these risk factors.

In some embodiments, the extended release dosage form comprising a selective serotonin 5-HT₃ antagonist is effective for prophylaxis or treatment of PONV/PDNV for surgical patients administered postoperative opioids for analgesia. Such opioids can include, for example, codeine, morphine, thebaine, oripavine, diacetyl morphine, dihydrocodeine, hydrocodone, hydromorphone, nicomorphine, oxycodone, oxymorphone, fentanyl, α-methyl fentanyl, alfentanil, sufentanil, remifentanil, meperidine, buprenorphine, etorphine, methadone, and tramadol.

### EXAMPLES

### Example 1

1.A SR-Coated Fumaric Acid Crystals: 40-80 mesh fumaric acid crystals (3750 g) were charged into a Glatt GPCG 5 fluid-bed coater equipped with a 9″ bottom spray Wurster insert, 10″ column length and 16 mm tubing. The fumaric acid crystals were coated with a solution (6% solids) of 250 g of ethylcellulose (EC-10: Ethocel Premium 10 cps) and 166.7 g of polyethylene glycol (PEG 400) at an EC-10/PEG 400 ratio of 60/40, dissolved in 98/2 acetone/water (6528.3 g), for a coating weight of up to 10% by weight. The processing conditions were as follows: atomization air pressure: 2.0 bar; nozzle diameter: 1.00 mm; bottom distribution plate: B with 15 gauge 100 mesh screen; spray/shake interval: 30 s/3 s; product temperature maintained at 35±1° C.; inlet air volume: 155-175 cubic feet per minute (cfm) and a spray rate increasing from about 8 to 30 g/min.

Fumaric acid crystals were also coated as described above using different ratios of ethylcellulose and PEG. More specifically, fumaric acid crystals were coated with a solution of EC-10/PEG 400 at a ratio of either 75/25 or 67.5/32.5, providing a coating weight of up to 10% by weight for each ratio.

1.B Ondansetron Hydrochloride IR Beads: Povidone (PVP K-29/32; 23 g) was slowly added to 50/50 water/Denatured Alcohol 3C, 190 Proof (3699.4 g), with mixing until dissolved. Ondansetron hydrochloride dihydrate (197.2 g) was slowly added to the povidone binder solution until the ondansetron hydrochloride was dissolved. SR-coated fumaric acid crystals (3000 g) obtained from Example 1.A, above, were coated in the Glatt GPCG 5 with the ondansetron solution (5% solids) while maintaining the product temperature at 40±1° C.; inlet air volume at 180-195 cfm and with a spray rate increasing from about 8 to 15 g/min. The resulting drug-layered beads were provided with a protective seal-coat of Opadry Clear (hypromellose 2910; 3 cps) (2% coating weight) to form IR beads.

1.C Ondansetron Hydrochloride TPR Beads: Ondansetron hydrochloride IR beads (2800 g) from Example 1.B were coated by spraying a solution in 98/2 acetone/water (6% solids) of EC-10/hydroxypropylmethyl cellulose (HPMCP; HP-55)/triethyl citrate (TEC) at a ratio of 45.5/40/14.5, and dried in the Glatt for about 10 minutes at 60° C. to drive off excess residual solvent, to provide a coating weight of up to 50%. The dried beads were sieved to discard any “doubles” formed.

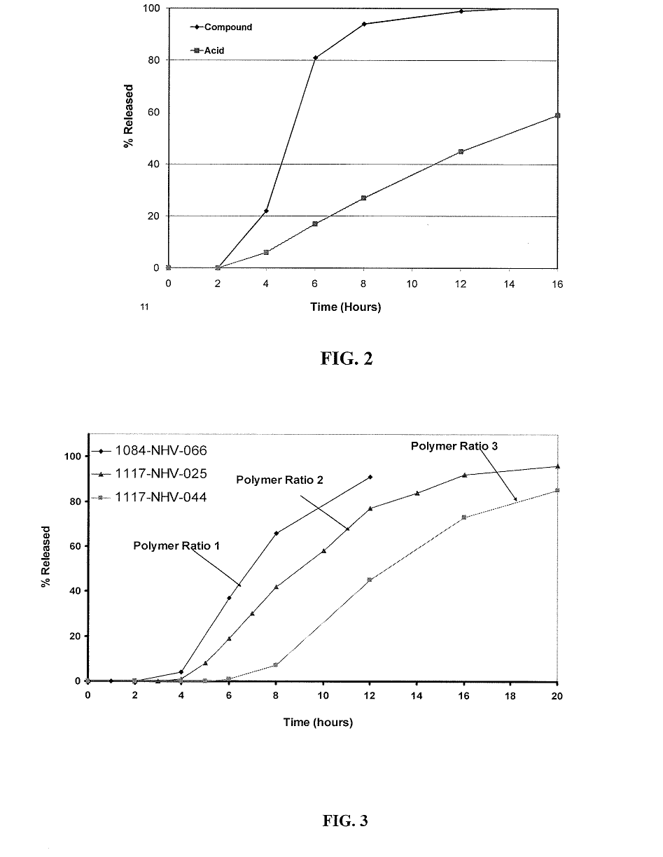

FIG. 2 shows the release profiles of both fumaric acid and ondansetron from TPR beads comprising SR-coated acid crystals. More specifically, the TPR Beads evaluated in FIG. 2 have the following composition:

| | | Coating Weight |
|---|---|---|
| | Composition | (wt. %) |
| core | fumaric acid crystals | N/A |
| SR Layer | EC-10/PEG 400 | 10 |
| | (67.5/32.5) | |
| Drug Layer | ondansetron HCl/PVP | 6 |
| | (90/10) | |
| TPR Layer | EC-10/HP-55/TEC | 50 |
| | (45.5/40/14.5) | |

Although the ondansetron release is significantly faster than the fumaric acid release, it will be apparent to a person skilled in the art that by decreasing the thickness of the barrier-coat (SR layer) on the fumaric acid crystals and/or additionally applying a SR layer under the TPR layer to sustain the drug release, the release profiles for both ondansetron and fumaric acid can be synchronized.

### Example 2

2.A Fumaric Acid-Containing Cores: Hydroxypropyl cellulose (Klucel LF, 53.6 g) was slowly added to 90/10 190 proof alcohol/water at 4% solids, with rigorous stirring until dissolved, and then fumaric acid (482.1 g) was slowly added and stirred until dissolved. A Glatt GPCG 5 equipped with a 9″ bottom spray Wurster insert, 10″partition column was charged with 3750 g of 25-30 mesh sugar spheres. The sugar spheres were layered with the fumaric acid solution while maintaining the product temperature at about 33-35° C. and at a spray rate of 8-60 mL/min. The acid cores were dried in the Glatt unit for 10 min to drive off residual solvent/moisture and sieved through 40-80 mesh screens.

2.B SR-coated Fumaric Acid-Containing Cores: Following the procedures of Example 1.A, fumaric acid cores (3750 g) from Example 2.A were coated with a solution of EC-10 mixed with either PEG 400 (B.1) at a ratio of 60/40 or TEC (B.2) at a ratio of 90/10 as the plasticizer, dissolved in 98/2 acetone/water (6% solids), providing a coating weight of 10%.

2.0 Ondansetron Hydrochloride IR Beads: Ondansetron hydrochloride IR beads (B.1 and B.2) were prepared as described in Example 1.B by coating the SR-coated fumaric acid-containing cores of Example 2.B with a solution of ondansetron hydrochloride dihydrate/povidone (90/10) at a drug load of 4 wt. % (as ondansetron base). The resulting drug-layered beads were provided with a protective seal-coat with Pharmacoat 603 (hypromellose 2910; 3 cps) for a weight gain of 2%.

2.D Ondansetron Hydrochloride SR Beads: Ondansetron hydrochloride IR beads (1080 g) from Example 2.0 were SR coated by spraying a solution of EC-10 mixed with either PEG 400 (D.1) at a ratio of 60/40 or TEC (D.2) as the plasticizer at a ratio of 90/10, dissolved in 98/2 acetone/water (7.5% solids), and dried in the Glatt at the same temperature for 10 minutes to drive off excess residual solvent, providing a coating weight of 10%. The dried beads were sieved to discard any doubles, if formed.

2.E Ondansetron Hydrochloride TSR Beads: Ondansetron hydrochloride SR beads (D.1 and D.2) from Example 2.D, were further coated with a TPR coating of EC-10/HP-55/TEC at three ratios: 45.5/40/14.5 (E.1—lot# 1084-066), 50.5/35/14.5 (E.2—lot# 1117-025) and 60.5/25/14.5 (E.3—lot# 1117-044) dissolved in 90/10 acetone/water (7.5% solids), at coating weights of up to 50%. The resulting TSR beads were dried in the Glatt to drive off residual solvent and sieved through a 18 mesh screen. FIG. 3 shows the release profiles of ondansetron hydrochloride from TSR beads coated with EC-10/HP-55/TEC at three different ratios. More specifically, FIG. 3 shows the release profiles for the following formulations presented in Table 1:

TABLE 1
Compositions of Ondansetron TPR Beads

| | |
|---|---|
| Composition | Coating Weight (%) |

E.1
Lot# 1084-066

| | | |
|---|---|---|
| Core | 25-30 mesh sugar spheres | N/A |
| Acid Layer | fumaric acid/Klucel | 6.0 |
| | (90/10) | |
| SR Layer | EC-10/PEG 400 | 10 |
| | (60/40) | |
| Drug Layer | ondansetron HCl/PVP | 5 (no seal coat) |
| (4% ondansetron base) | (90/10) | |
| SR Layer | EC-10/TEC | 10 |
| | (90/10) | |
| TPR Layer | EC-10/HP-55/TEC | 50 |
| | (45.5/40/14.5) | |

E.2
Lot# 1117-025

| | | |
|---|---|---|
| Core | 25-30 mesh sugar spheres | N/A |
| Acid Layer | fumaric acid/Klucel | 6 |
| | (90/10) | |
| SR Layer | EC-10/TEC | 10 |
| | (90/10) | |
| Drug Layer | ondansetron/Klucel LF | 7 |
| (4% ondansetron base) | (90/10) | |
| SR Layer | EC-10/TEC | 10 |
| | (90/10) | |
| TPR Layer | EC-10/HP-55/TEC | 50 |
| | (50.5/35/14.5) | |

E.3
Lot# 1117-044

| | | |
|---|---|---|
| Core | 25-30 mesh sugar spheres | N/A |
| Acid Layer | fumaric acid/PVP | 6 |
| | (90/10) | |
| SR Layer | EC-10/TEC | 10 |
| | (90/10) | |
| Drug Layer | ondansetron/Klucel LF | 7 |
| (4% ondansetron base) | (90/10) | |
| SR Layer | EC-10/TEC | 10 |
| | (90/10) | |
| TPR Layer | EC-10/HP-55/TEC | 50 |
| | (60.5/25/14.5) | |

### Example 3

3.A Ondansetron Hydrochloride RR Beads at a drug load of 10%: Hydroxypropylcellulose (Klucel LF from Aqualon, 33 g) was slowly added to 50/50 water/Denatured Alcohol 3C, 190 Proof (2500 g each) while mixing to dissolve. Ondansetron hydrochloride dihydrate (300 g) was slowly added to the above binder solution until the drug was dissolved. 60-80 mesh sugar spheres (2607 g) were coated with the drug solution (5% solids) in a Glatt GPCG 5 to provide a drug content of 10 wt. % (as ondansetron base) under the following conditions: air distribution plate: B with 100 mesh screen; nozzle diameter: 1 mm; partition height: 10″; 9″ bottom spray Wurster insert; product temperature at 36-37° C.; inlet air volume at 60-65 cfm and increasing spray rate from about 20-25 g/min. The resulting drug-layered beads were provided with a protective seal-coat of Pharmacoat 603 (hypromellose 2910; 3 cps) (2% weight gain) to form RR beads. The RR beads were dried in the Glatt unit for 10 min to drive off residual solvent/moisture and sieved through 40-80 mesh screens. More than 90% of the IR beads were in the particle size range of 100-350 μm.

3.B Ondansetron Hydrochloride RR Granules at a drug load of 10%: Fumaric acid (270 g), Klucel LF (120 g), and ondansetron HCl (600 g) were slowly added to a 50/50 mixture of Denatured 190 Proof Ethyl Alcohol and water (5000 g each) in a stainless steel tank, with agitation until dissolved. A Glatt GPCG 5 equipped with a top spray Wurster insert was pre-heated for not less than 30 min and charged with spray dried lactose (Fast Flo Lactose; 2130 g), microcrystalline cellulose (MCC, Avicel PH102; 2400 g); Crospovidone (XL-10; 480 g), which were then granulated while spraying with the ondansetron solution at a rate of 25-100 g/min under the following conditions: granulating bowl: GPCG 5 with top spray; nozzle tip: 1.2 mm; inlet air temperature: 55° C.; air flow target: 80 cfm; atomization air pressure: 2.0 bar; product temperature target: 50° C. The granulation was dried at 55° C. to a loss on drying (LoD) value of <2%. The granules were sieved through a 20 mesh screen and blended with magnesium stearate (10 g per 5000 g of granules) in a 0.5 cu.ft. V blender rotating at 21 RPM for 5 minutes.

3.C Fumaric Acid-Containing Cores: 25-30 mesh sugar spheres (3750 g) were layered with fumaric acid (482.1 g) from a solution (4% solids) of Klucel LF (53.6 g) as described in Example 2.A above, to achieve a fumaric acid load of 11.25% by weight. The fumaric acid-containing cores were dried in the Glatt unit for 10 min to drive off residual solvent/moisture, and sieved through 20-30 mesh screens.

3.D SR-coated Fumaric Acid Cores: The fumaric acid-containing cores (3750 g) from Example 3.0 were coated with a solution of 177.6 g of ethylcellulose (EC-10) and 19.7 g of triethyl citrate (TEC) at a ratio of 90/10, dissolved in 95/5 acetone/water (7.5% solids), providing a coating weight of 5%.

3.E Ondansetron Hydrochloride IR Beads: IR beads of ondansetron hydrochloride dihydrate with a drug load of 10% by weight were produced by spraying a solution (5% solids) of ondansetron hydrochloride dihydrate (402.8 g) and Klucel LF (44.3 g) dissolved in a 50/50 ethanol/water mixture (4247.4 g each), onto SR-coated fumaric acid cores (3500 g) from Example 3.D, above, in a Glatt GPCG 5 under the following conditions: air distribution plate: B with 15 gauge 100 mesh screen; nozzle diameter: 1 mm; partition height: 10″; 9″ bottom spray Wurster insert; product temperature at 34±1° C.; inlet air volume at 150 cfm; atomization air pressure −1.5 bar; and an increasing spray rate of from 8 to 30 mL/min. The resulting drug-layered beads were provided with a protective seal-coat of Pharmacoat 603 (hypromellose 2910; 3 cps) (2% weight gain) to form IR beads with an ondansetron content of 10 wt. % (as ondansetron base). The resulting IR beads were dried in the Glatt unit for 10 min to drive off residual solvent/moisture, and sieved to discard oversized and undersized particles.

3.F-1 Ondansetron Hydrochloride TPR Beads at 15% Coating: Ondansetron hydrochloride IR beads (3500 g) from Example 3.E, above, were coated with a TPR coating of ethylcellulose (389.1 g), HP-55 (135.9 g) and TEC (92.6 g) (ratio: 63/22/15) dissolved in 90/10 acetone/water by spraying the solution (18% solids) to a coating weight of 15%, and dried in the Glatt at the coating temperature for 10 minutes to drive off excess residual solvent. The dried beads were sieved to discard any doubles, if formed.

3.F-2 Ondansetron Hydrochloride TPR Beads at 10% Coating: Ondansetron hydrochloride IR beads (3500 g) from Example 3.E, above, were coated with a TPR coating of ethylcellulose (245.0 g), HP-55 (85.6 g) and TEC (58.3 g) (ratio: 63/22/15) dissolved in 90/10 acetone/water by spraying the solution (18% solids) to a coating weight of 10%, and dried in the Glatt at the coating temperature for 10 minutes to drive off excess residual solvent. The dried beads were sieved to discard any doubles, if formed.

3.G-1 Ondansetron Hydrochloride MR Capsules (PF391EA0001): Rapid Release Granules (100.0 mg of RR granules of lot# PE391EA0001) prepared as described in Example 3.B, above, and TPR beads (166.2 mg of TPR beads of lot# PE392EA0001) prepared as described in Example 3.F-1, above, were filled into size ‘0’ hard gelatin capsules to produce Test Formulation A: MR Capsules, 20 mg (8 mg RR+12 mg TPR (T_{80%}˜8 hrs) where T_{80%} refers to the total time to achieve 80% of the drug released).

3.G-2 Ondansetron Hydrochloride MR Capsules (PF392EA0001): Rapid Release Granules (100.0 mg of RR granules of lot# PE391EA0001) prepared as described in Example 3.B, above, and TPR beads (221.6 mg of TPR beads of lot# PE292EA0001) prepared as described in Example 3.F-1, above, were filled into size ‘0’ hard gelatin capsules to produce Test Formulation B: MR Capsules, 24 mg (8 mg RR+16 mg TPR (T_{80%}˜8 hrs)).

3.G-3 Ondansetron Hydrochloride MR Capsules (PF379EA0001): Rapid Release Granules (100.0 mg of RR granules of lot# PE391EA0001) prepared as described in Example 3.B, above, and TPR beads (234.6 mg of TPR beads of lot#PE393EA0001) prepared as described in Example 3.F-2, above, were filled into size ‘0’ hard gelatin capsules to produce Test Formulation C: MR Capsules, 24 mg (8 mg RR+16 mg TPR (T_{80%}˜12 hrs)).

### Example 4

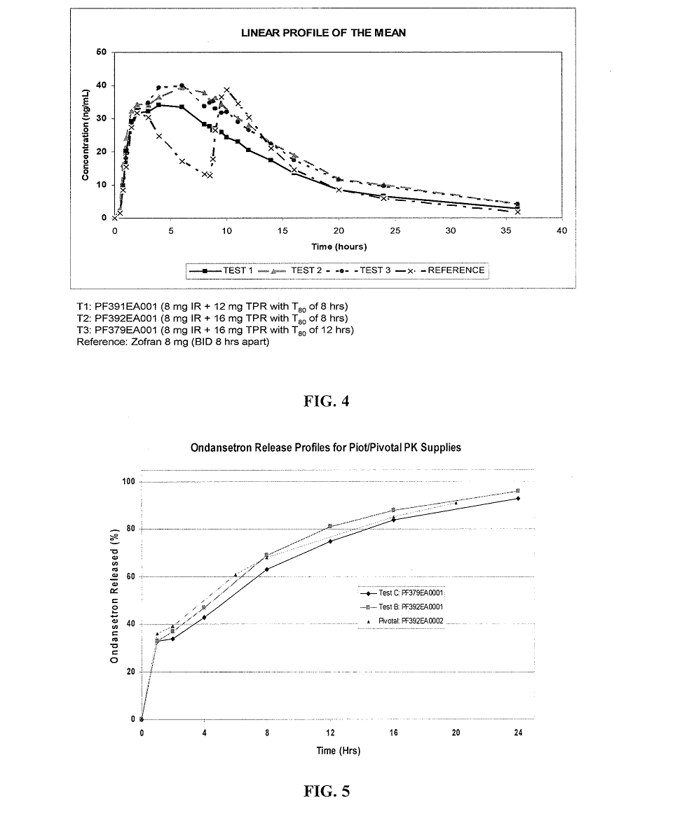

4.A Pilot PK Study (ODO-P7-220): Ondansetron Hydrochloride MR Capsules vs. Zofran: A 4-arm crossover pilot PK (pharmacokinetics) study was conducted which included 12 male, healthy volunteers aged 18 to 55 years with a wash-out period of 7 days. Each volunteer was dosed with 250 mL of still mineral water and a single Test Formulation: Test Formulation A (20 mg; PF391EA0001); Test Formulation B (24 mg; PF392EA0001), Test Formulation C (24 mg; PF379EA0001) at 8 AM; or two Zofran® (8 mg) at 8 AM and 4:30 PM after fasting overnight (at least 12 hrs; lunch was served at 11 AM). Blood samples were drawn at time 0 (pre-dose), 20 min, 40 min, 1 hr, 1.5 hrs, 2 hrs, 3 hrs, 4 hrs, 6 hrs, 8.5 hrs (before second dose), 9 hrs 10 min, 9.5 hrs, 10 hrs, 10.5 hrs, 11.5 hrs, 12.5 hrs, 14.5 hrs, 17 hrs, 20 hrs, 22 hrs, 24 hrs and 36 hrs. FIG. 4 shows the mean plasma concentration-time profiles achieved. The PK parameters (actual as well as dose normalized) are presented in Table 2. The relative bioavailability compared to the 8 mg IR (Zofran) bid reference was approximately 0.85 for all test formulations (Test Formula A, B, and C) at the end of 24 hours.

TABLE 2
PK Parameters from Pilot PK Study

| | Test-A | | Test-B | Test-C | |
|---|---|---|---|---|---|
| PK Parameters | (Ondansetron 20 mg | | (Ondansetron 24 mg | (Ondansetron 24 mg | |
| Mean (90% C.I.) | PF391EA0001)/ | | PF392EA0001)/ | PF379EA0001)/ | |
| Cₘₐₓ (μg/mL) | 89% | (84-95%) | 107% (100-114%) | 104% | (97-111%) |
| AUC₀₋ₜ | 109% | (102-117%) | 132% (132-152%) | 137% | 128-146%) |
| (μg · hr/mL) | | | | | |
| AUC_{0-inf} | 113% | (105-122%) | 150% (139-161%) | 145% | (135-146%) |
| (μg · hr/mL) | | | | | |
| | Dose Normalized PK Parameters | | | | |
| Relative | 92% | (86-98%) | 98% (92-104%) | 95% | (89-101%) |
| Bioavailability (90% | | | | | |
| Confidence Interval) | | | | | |

4.B Ondansetron RR Granules (PE391EA0004): Fumaric acid (3.60 kg), Klucel LF (1.60 kg), and ondansetron HCl (8.00 kg) were slowly added to a 50/50 mixture of Denatured 190 Proof Ethyl Alcohol and water (66.7 kg each) in a 100-gallon stainless steel tank equipped with a propeller mixer, and agitated at about 850 rpm until dissolved. A Glatt GPCG 120 equipped with a top spray Wurster insert and a 32″ Granulation bowl was pre-heated to a process air temperature of 76° C. and air volume of 600 cfm and charged with lactose monohydrate (28.4 kg), microcrystalline cellulose (MCC; Avicel PH102, 32.0 kg), and crospovidone (XL-10; 6.4 kg) and granulated while spraying the drug solution at a rate of 0.45-0.60 kg/min under the following conditions: top spray nozzle tips (3): 1.8 mm; inlet air temperature: 71-86° C.; air flow target: 500-900 cfm; atomization air pressure: 2.0 bar; product temperature target: 36-37° C. The granulation was dried at a process air temperature of 77° C. and an air volume of 800 cfm, until a loss on drying value of <2% was obtained. The granules were then sieved through a 30 mesh screen (oversized material was discarded) and blended with magnesium stearate (0.17 kg per 77.8 kg of beads) in a 10 cu.ft. V blender rotating at 17.5 rpm for 6 minutes, then discharged into 41 gallon drums double-lined with polyethylene bags.

4.0 Fumaric Acid SR Beads (PE363EA0001): Klucel LF (1.00 kg) and fumaric acid (8.50 kg) were slowly added to a mixture of Denatured 190 Proof SD 3 Ethyl Alcohol (205.2 kg) and water (22.8 kg) in a 100-gallon stainless steel tank equipped with a propeller mixer, while agitating at about 1000 rpm, until the Klucel and fumaric acid were dissolved. A Glatt GPCG 120 equipped with an 18″ bottom spray Wurster insert; air distribution plates (inner: G 1-122-00017-3; outer: C 1-122-00022-4) and 200 mesh product support screen, was pre-heated to a process air temperature of 53° C. and air volume of 600 cfm and charged with 25-30 mesh Sugar Spheres (66.5 kg), which were coated by spraying the fumaric acid/Klucel solution at 150-600 g/min under the following conditions: bottom spray nozzle tip: 3 mm; inlet air temperature: 60-70° C.; air flow volume: 570-730 cfm; atomization air pressure: 2.0 bar; product temperature target: 32-34° C.

Upon completion of fumaric acid layering, the spray system was rinsed with ethyl alcohol (˜200 g), the fumaric acid layered beads were sprayed with a solution (7.4% solids) of ethylcellulose (3.60 kg of Ethocel Standard 10 Premium) and triethyl citrate (0.40 kg) dissolved in 95/5 acetone (46.9 kg)/water (2.5 kg) in a 30 gallon stainless steel mixer, agitated at 850 rpm. The resulting fumaric acid SR beads were dried at a process air temperature of 43° C. and an air volume of 700 cfm for 10 min to drive off residual solvents including moisture. The beads were then sieved through a 30 mesh screen (oversized material was discarded) and blended with magnesium stearate (0.17 kg per 77.8 kg of beads) in a 10 cu.ft. V blender rotating at 17.5 rpm for 6 minutes, then discharged into 41 gallon drums double-lined with polyethylene bags.

4.D Ondansetron TPR Beads (PE392EA0005): IR beads of ondansetron hydrochloride dihydrate with a drug load of 10% by weight (as ondansetron base) were produced by spraying a solution (5% solids) of ondansetron hydrochloride dihydrate (3.6 kg) and Klucel LF (0.40 kg) dissolved in a 50/50 Denatured 190 Proof SD 3C Ethyl alcohol/water mixture (38.0 kg each), onto fumaric acid SR beads (31.3 kg) from Example 4.C, above, in a the Glatt GPCG 120 (equipped as described in Example 4.C) under the following conditions: process air temperature: 75-80° C.; product temperature: 34±1° C.; inlet air volume at 450-500 cfm; and at a spray rate increasing from 100 to 400 g/min. The resulting drug-layered beads were provided with a protective seal-coat of Pharmacoat 603 (hypromellose 2910; 3 cps) (2% weight gain) to form ondansetron-containing IR beads.

Ethylcellulose (2.50 kg), hypromellose phthalate (0.90 kg HP-55) and triethyl citrate (0.60 kg) were slowly added to a mixture of acetone (33.8 kg) and purified water (2.16 kg) in a 30 gallon stainless steel tank, while agitating at approximately 850 rpm, until the HP-55 and triethyl citrate dissolved. Ondansetron hydrochloride IR beads prepared in Example 4.D, above, were sprayed with a TPR coating solution (10% solids; 63/22/15 ethylcellulose/HP-55/TEC) at 100-300 g/min, then dried in the Glatt at a process air temperature of 45° C. and an air volume of 500 cfm for 10 minutes to drive off excess residual solvent. A TPR coating weight of 10% was obtained. The dried beads were sieved with 18 and 30 mesh screens, and any doubles formed were discarded.

4.E Ondansetron Hydrochloride MR Capsules (PF392EA0002): Rapid Release Granules (100.0 mg of RR granules of lot# PE391EA0004) of Example 4.B and TPR beads (221.6 mg of TPR beads of lot# PE292EA0005) of Example 4.D were filled into white opaque size ‘1’ hard gelatin capsules (shell weight: 76 mg with a capsule weight of 397.6 mg) to produce 25,000 capsules of a pivotal CTM Test Formulation: MR Capsules, 24 mg (8 mg RR+16 mg TPR (T80%˜8 hrs)). FIG. 5 shows the ondansetron release profile of PF392EA0002 in comparison to the POC (proof of concept) CTM supplies (Test B—PF392EA0001 and Test C—PF279EA001).

A PK/PD model based on ondansetron exposure and the corresponding onset and duration of antiemetic responses for Zofran®, an IR ondansetron formulation was used to compare ondansetron bioavailability for three modified-release formulations of ondansetron and Zofran® (bid). The model shows similar drug exposures (area under plasma concentration curve, AUC) between the modified release formulations of the present invention, and those of bid Zofran®.

As per regulatory requirements, a single dose (qd 24 mg) food effect study of modified release formulations of the present invention, and a comparative PK study of inventive formulations (qd 24 mg) versus Zofran under the approved individual dosing regimens for each indication being pursued were conducted, and additional pharmacokinetic data were collected according to the specific dose and frequency of administration of each indication for which Zofran® is currently marketed as discussed below. The products were administered either under fed or fasting conditions based on the approved individual dosing regimens for each indication. The safety profile of each treatment was also assessed by recording the nature, severity, frequency, duration and relation to the treatment of any adverse event.

### Example 5

5.A Pivotal Food Effect Study: A 2-arm, single dose, crossover food effect study in 20 healthy subjects, and 17 subjects (10 female and 7 male) completed the dosing sequence. The inventive modified-release formulation, in the presence of a high-fat diet exhibited a slightly decreased Cₘₐₓ, and delayed Tₘₐₓ in comparison to its behavior in the fasted state. However, overall exposure (AUC) was similar between the fasted and the fed states.

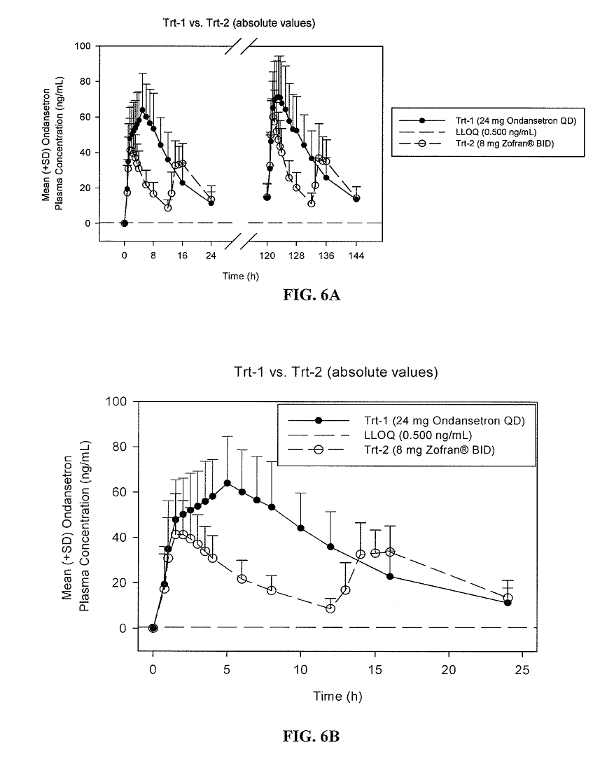

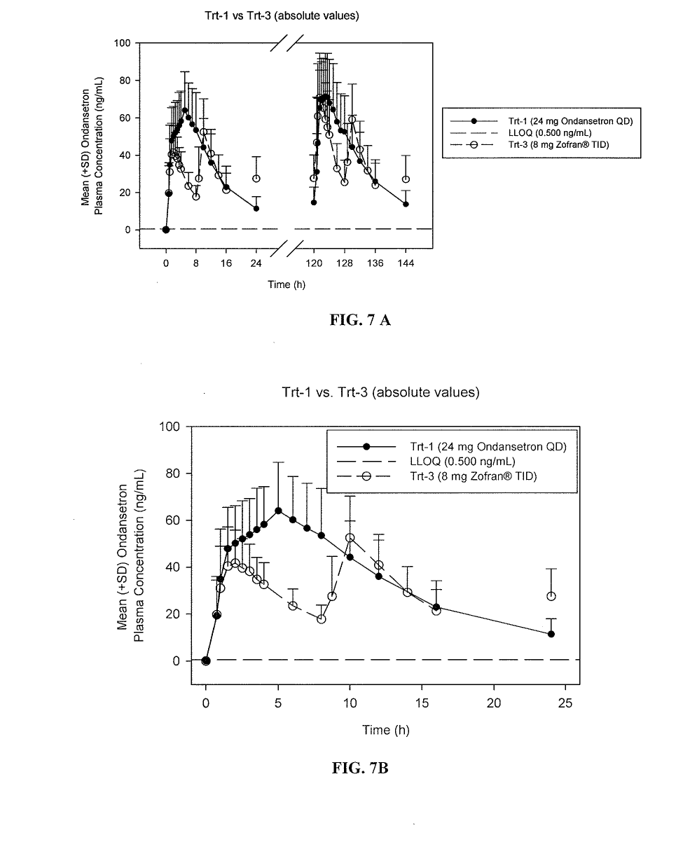

5.B Pivotal Single and Multiple dose PK Study: A single center, parallel group (4 groups each with 30 subjects), 4-treatment (7 or 8 subjects per treatment), single and multiple dose PK study of IR ondansetron (Zofran®) and inventive modified release ondansetron formulations in 120 healthy volunteers was carried out. The objective of this study was to evaluate the PK properties of ondansetron administered as an inventive modified-release formulation (“MR”; qd, 24 mg) as well as an IR formulation (Zofran®, 8 mg) after single and multiple oral dose administration, for the prevention of nausea and/or vomiting following surgery in patients at moderate to high risk of PONV/PDNV. Each dose of ondansetron was orally administered with approximately 240 mL of water. Treatment 1: a 24 mg dose of ondansetron MR dosage form, once-daily in the morning, over 6 consecutive days. Treatment 2: an 8 mg dose of IR ondansetron (1 Zofran® tablet) twice daily, 12 hrs apart (morning and evening), over 6 consecutive days. Treatment 3: an 8 mg dose of ondansetron (1 Zofran® tablet) thrice daily, 8 hrs apart (morning, afternoon, and evening), over 6 consecutive days. Treatment 4: the following doses of ondansetron were administered in the morning: Day 1: a single dose of Zofran®, Day 2: one placebo capsule; Day 3: a single 16 mg dose of ondansetron (2×8 mg Zofran®); Day 4 and 5: two placebo capsules on each day; and Day 6: a single 24 mg dose of ondansetron (3×8 mg Zofran® tablet). Blood samples (2×6 mL) were collected by venipuncture in pre-cooled Vacutainer tubes containing K2 EDTA at appropriate pre- and post-dose timings (preselected for each treatment) and analyzed using a validated HPLC/MS assay with an analytical range of approximately 0.5 to 300 μg/mL. A single and multiple dose, parallel group study of Zofran® IR tablets, 8 mg bid versus the inventive MR dosage form (qd, 24 mg) in 120 healthy volunteers (males+females), 117 subjects, completed the study of dosing requirements. Single and repeated oral administrations of 24 mg ondansetron MR dosage form once daily resulted in similar rate and extent of exposure as 8 mg Zofran® administered thrice daily. FIGS. 6A and 6B show the ondansetron plasma concentrations after once-daily administration of a 24 mg ondansetron MR dosage form versus 8 mg Zofran® administered twice daily, and FIGS. 7A and 7B show the ondansetron plasma concentrations after once-daily administration of a 24 mg ondansetron MR dosage form versus 8 mg Zofran® administered thrice daily. Based on the above results, the 24 mg ondansetron MR dosage form is expected to result in similar efficacy as 8 mg Zofran® administered thrice daily.

Relative Bioavailability: Ondansetron QD 24-mg vs. Zofran® BID/TID

Total exposures of ondansetron (AUC₀₋₂₄) following administration of Zofran® BID and TID on Day 6 were 21% and 29% higher, respectively, than those observed on Day 1, suggesting minimum accumulation following repeated BID and TID dosing for 6 days. Total exposure of ondansetron (AUC₀₋₂₄) from 24 mg Ondansetron QD on Day 6 was approximately 12% higher than that observed on Day 1 (936 vs. 825 ng·h/mL, respectively), suggesting minor accumulation following repeated dosing (FIG. 6A). Consistent with the total daily dose of 24 mg Ondansetron QD treatment as compared to the 8 mg Zofran® BID treatment, geometric least-square means (LSM) of AUC₀₋₂₄ following oral administrations of the 24 mg Ondansetron QD treatment were approximately 40-50% higher than those observed for the 8 mg Zofran® BID treatment (FIGS. 6A and 6B). Ratios of LSM for AUC₀₋₂₄ of ondansetron on Day 1 and 6 following oral administrations of the 24 mg Ondansetron QD treatment compared to the 8 mg Zofran® TID treatment were 98.4% and 86.6%, with 90% CIs falling within 80-125% on Day 1 (FIGS. 7A and 7B). Although the 90% CIs for Day 6 did not fall within 80-125%, the ratio of LSM for AUC₀₋₂₄ was nevertheless within 80-125%. Ratios of LSM for Cₘₐₓ of ondansetron on Day 1 and 6 were 108% and 94.0%, respectively, with both 90% CIs falling within 80-125%. These results suggest that single and repeated oral administrations of 24 mg Ondansetron QD resulted in similar rate and extent of exposure as the 8 mg Zofran® TID treatment. Based on the above results, the 24 mg ondansetron MR dosage form is expected to result in similar efficacy as 8 mg Zofran® administered thrice daily.

Relative Bioavailability: Ondansetron QD 24-mg vs. Zofran® Single Doses

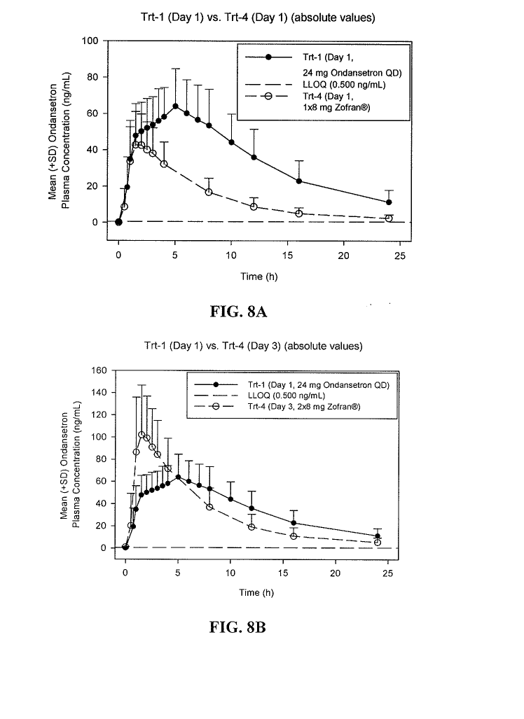

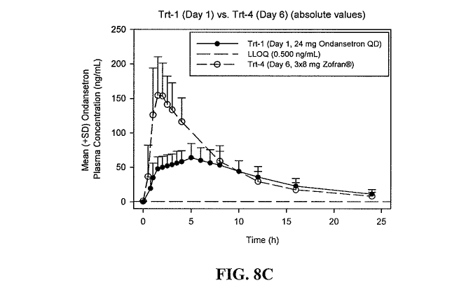

Table 3 shows the relative bioavailability of Ondansetron QD 24-mg vs. Zofran® single dose, 8-mg, 16-mg, 24-mg (Treatment-1 vs. Treatment-4). The corresponding plasma concentration—time curves on day 1, day 3 and day 6 (Treatment-1 vs. Treatment-4) are shown in FIGS. 8A, 8B, and 8C, respectively. Ratios of LSM for the AUC₀₋₂₄, AUC_{0-inf} and Cₘₐₓ of ondansetron following oral administration of Ondansetron QD 24-mg relative to 24 mg of Zofran® (Day 6) were 73.8%, 77.9% and 38.6%, respectively.

TABLE 3
Relative Bioavailability of 24 mg Ondansetron QD vs. 8 mg Zofran ® Single dose

| | | Geometric LSM | | | | |
|---|---|---|---|---|---|---|
| | | (Inter-Subject CV %) | | | Ratio of LSM (%) | |
| PK | | Treatment-1 | Zofran ® | | Treatment-1/Treatment-4 | |
| Parameter | Day | (24 mg Ondansetron QD | Single Dose | | (90% CI ) | |
| AUC₀₋₂₄ | Day 1 | 783 (34.9) | 321 | (40.2) | 244 | (210.7-282.8) |
| | Day 3 | NA | 661 | (44.5) | 118 | (100.1-140.0) |
| | Day 6 | NA | 1060 | (53.9) | 73.8 | (61.1-89.1) |
| AUC_{0-inf} | Day 1 | 889 (37.5) | 343 | (42.8) | 259 | (221.2-302.6) |
| | Day 3 | NA | 712 | (47.4) | 125 | (104.4-149.2) |
| | Day 6 | NA | 1060 | (53.9) | 73.8 | (61.1-89.1) |
| Cₘₐₓ | Day 1 | 61.1 (30.8) | 47.9 | (35.2) | 128 | (112.0-145.3) |
| | Day 3 | NA | 98.4 | (37.0) | 62.0 | (53.7-71.7) |
| | Day 6 | NA | 158 | (48.9) | 38.6 | (32.5-45.8) |

*Day 1 = 8 mg, Day 3 = 16 mg and Day 6 = 24 mg; NA = Not applicable

### Example 6

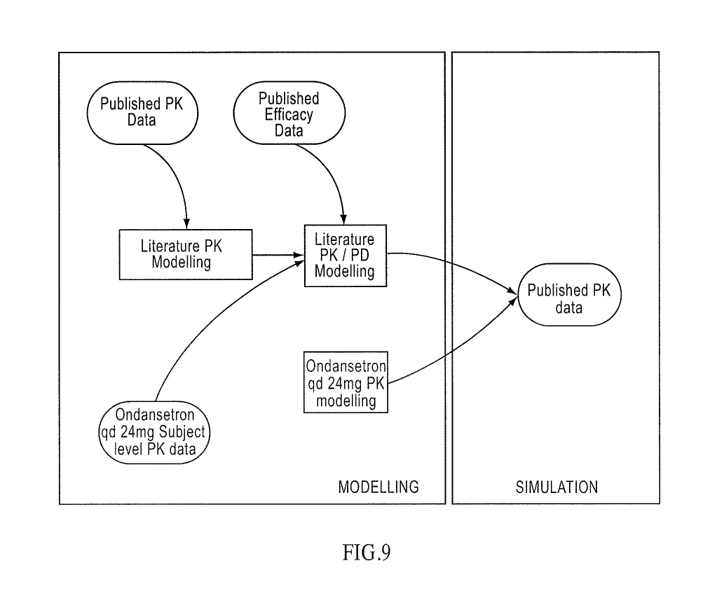

6.A Modeling PONV: A schematic representation of the model-based drug development approach is presented in FIG. 9. Exposure driven response assumptions were made (i.e., ondansetron has effect directly after administration so that the partial AUC (e.g. AUC_{0-1 hr}) was considered an appropriate effect predictor), and the model also considered the dose regimen, administration route (intravenous or peroral), when the dose was administered (i.e. pre-surgery, post-surgery), etc. Two separate models were established: (1) an incidence rate model using all data; and (2) an incidence-exposure model for (0-2 hr) and (0-24 hr) data only. Three incidence endpoints, nausea, emesis, and rescue medication were considered, which allow the modeling of all incidence endpoints simultaneously, and allow incidence to be inferred at any time interval post surgery. Several different exposure measures—AUCs until 15, 30, 45, 60 and 120 minutes post dose (adjusted for whether treatment was given pre-surgery or post-surgery), and concentration at midpoint of interval were tested. Demographic information about previous PONV for the patients was included in the model.

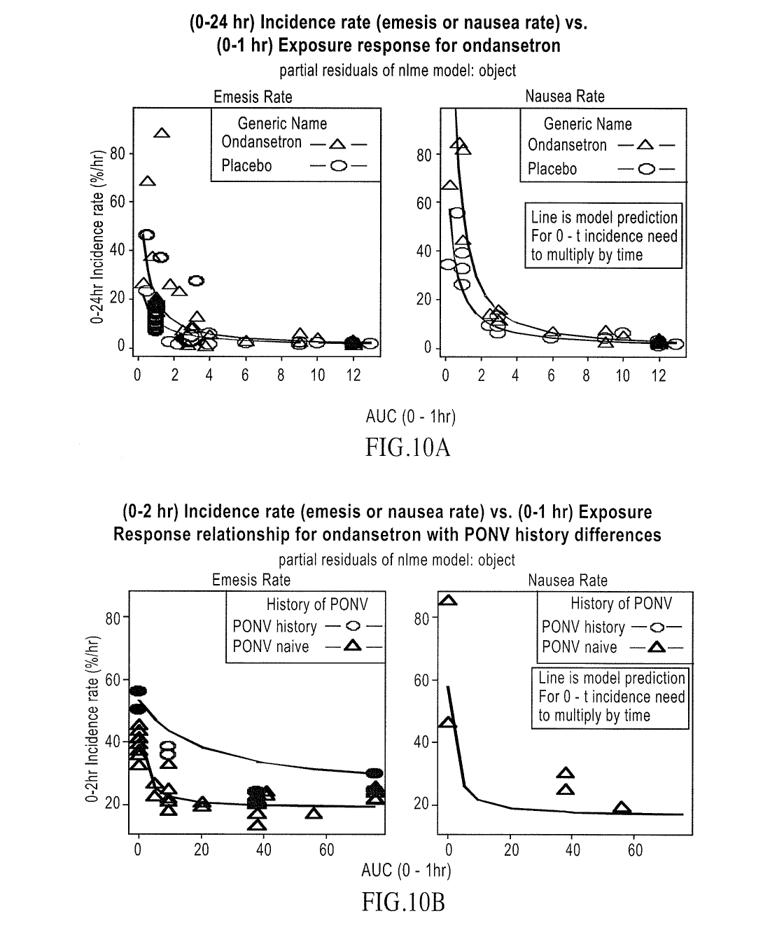

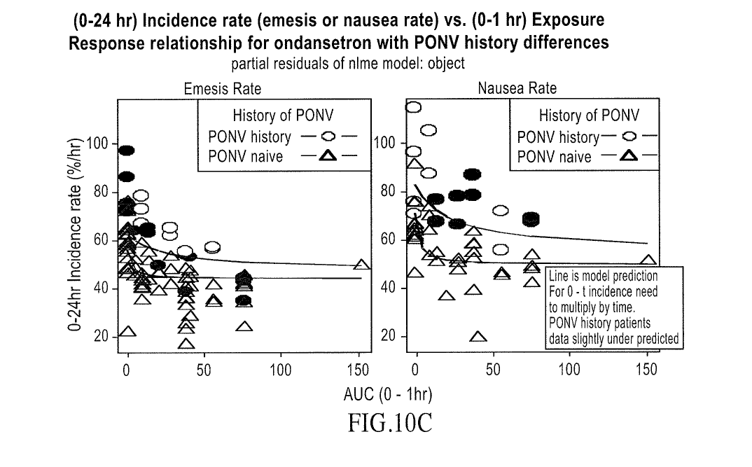

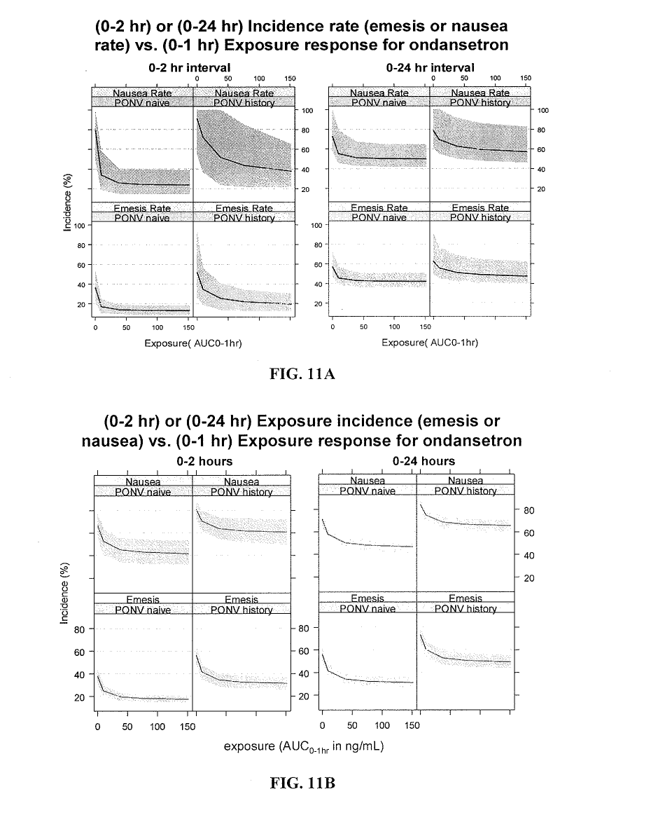

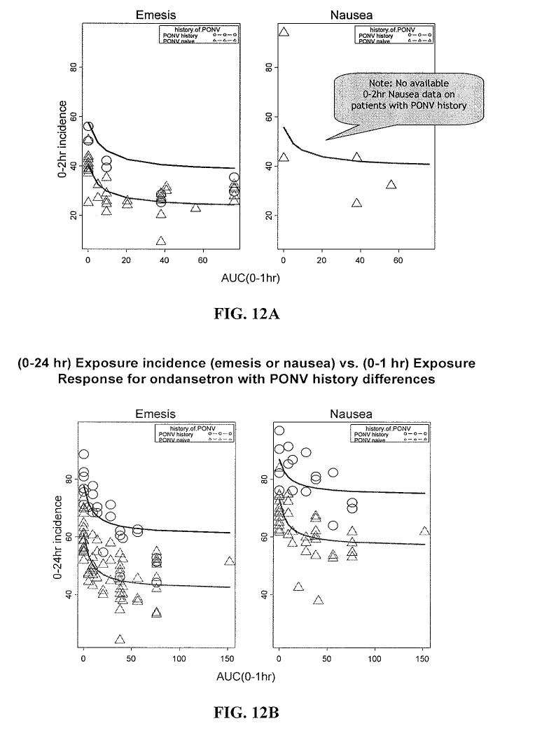

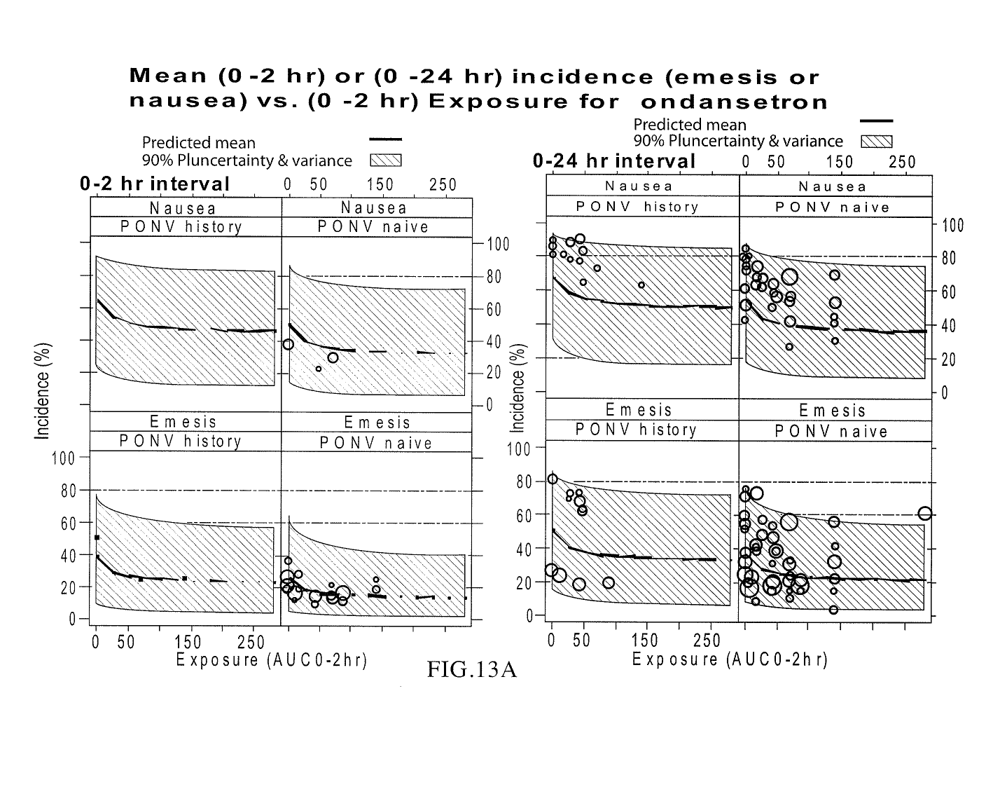

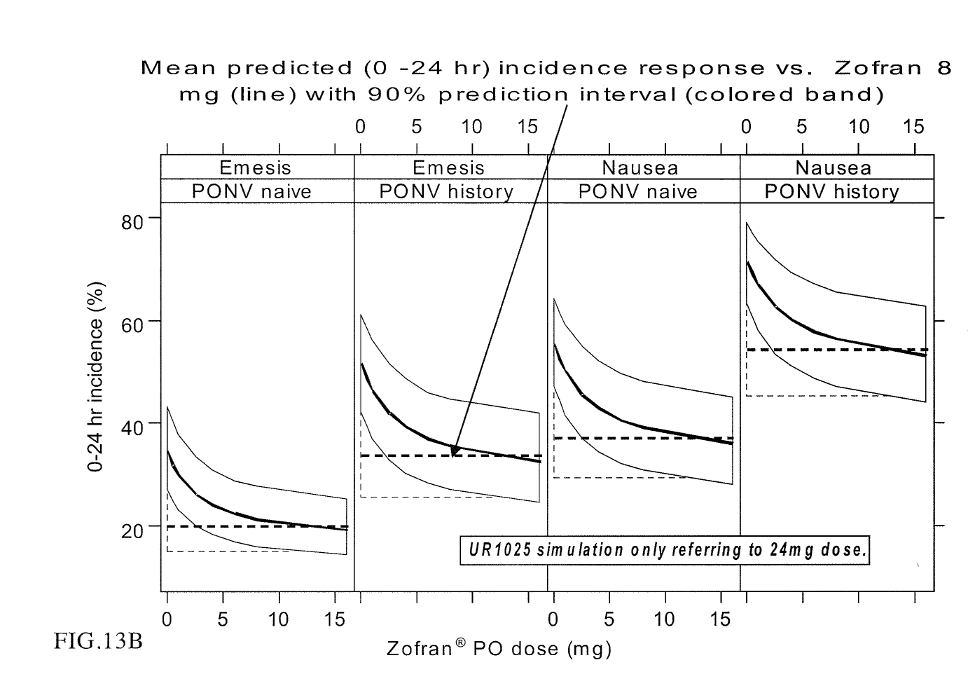

Simultaneous analysis of emesis and nausea incidence-rate/incidence-exposure: Modeling based on incidence-rate and incidence-exposure provided a useful and predictive model by examining the relationships between incidence-rate or incidence-exposure and AUC_{0-t hr} from different angles. Similar incidence rate profiles were obtained for each endpoint, i.e. nausea, emesis, and rescue medication incidence, thereby indicating that a single model is capable of predicting each endpoint. Examination of graphical representations of incidence endpoints (e.g., nausea, vomiting, or rescue medication) from the literature revealed that a typical PONV data set has timing overlap for measuring endpoints, i.e. 0-2 and 0-24 hours and the summary of incidence endpoints stratified by patient history of previous PONV indicated that these patients may be sensitive to PONV. It was also evident that nausea and vomiting data (but not rescue medication data) exhibit a clear exposure/response signal. PONV history patients may have different exposure/response relationships for the rescue medication endpoint. Rescue medication was excluded from the analysis as it added little value as evident from the preliminary analysis and the data were both sparse and diverse. FIGS. 10A, 10B, and 10C demonstrate the relationships between the (0-24 hr) incidence-rate and (0-2 hr) incidence-rate or (0-24 hr) incidence-rate with PONV history differences as a function of (0-1 hr) exposure-response, respectively. The conclusions are (1) the model fit across time post-surgery appears reasonable and (2) the model predicts ondansetron exposure response (0-2 hr) or (0-24 hr) relationships and PONV history differences reasonably well. Predictions of (0-2 hr) or (0-24 hr) incident-rate or exposure-incidence as a function of ondansetron (0-1 hr) exposure-response as AUC after administration (adjusted for whether treatment was given pre-surgery or post-surgery) are shown in FIG. 11A or 11B, which includes demographic information about previous PONV for the patients. The model predictions of incidence-exposure (e.g., exposure-nausea or exposure-emesis) as a function of exposure-response for (0-1 hr) after surgery are reasonably good, and accounts for PONV history (FIGS. 12A and 12 B). Predictions of PONY incidence response are shown in FIG. 13B. The uncertainty bands are moderate (see FIGS. 13A and 13B), which indicates that useful predictions can be made for the efficacy of administering modified-release ondansetron compositions according to the present invention. Unlike the incidence-rate model, the incidence-exposure model does not describe a different dose response in patients with history of PONV. The literature model suggests that ondansetron treatment results in an improved effect related to exposure.

### Example 7

7.A Clinical Trial Simulations: Simulations provide inferences about a future clinical trial and predict the probability of achieving non-inferiority in the next clinical trial of sample size n. Technical aspects are as follows:
Consider clinical trial design X, with sample size n;
Many parameter sets will be randomly drawn from the fixed effect estimates across parameter uncertainty to simulate, based on ondansetron PK exposure, the 0-2 hr and 0-24 hr incidence for particular future trials;
Using the simulated probability of an event based on the ondansetron PK exposure, n individual patient responses are simulated for design X;
Patient response: 0=no emesis, or 1=emesis;
The trial aim proportion of responders reflects a single representation of a model-based outcome of design X at sample size n;
The distribution of many simulated outcomes reflects the model-based predicted range of likely trial outcomes under design X and sample size n;
Various statistical tests can be applied on these results to test for, for example, non-inferiority criteria.

In order to perform simulations to provide inferences about future clinical trials, a response probability is drawn from the parameter uncertainty for each clinical trial. The 0-24 hr number of responders/non-responders are simulated across sample size. The expected responder fraction in either arm of each trial are calculated (i.e. p1, p2) where p1=fraction modified release (MR) ondansetron responders, p2=fraction IR ondansetron (Zofran®) responders. The lower limit (95% CI) of ratio p1/p2 is calculated and the proportion of trials where the lower limit exceeds the non-inferiority margin δ (δ should be in the 0.5-1 range to indicate non-inferiority) is reported. For example, the predicted mean complete responder (0-24 hr) ratio for a future trial of MR ondansetron vs. Zofran® 16 mg SD (both given pre-operatively) is about 1 for n=300 for nausea, nausea (PONV history), emesis and emesis (PONV history).

7.B Clinical Efficacy/Non-inferiority Study of PONV/PDNV: A randomized double-blind, placebo-controlled, multi-center study to evaluate the safety and efficacy of MR ondansetron capsules in post-operative nausea and vomiting in patients at moderate to high risk of PONV/PDNV (40% risk for PONV per the FDA guidelines) is conducted for 72 hrs following surgery. The patients receive one MR ondansetron capsule (24 mg capsule comprising RR and TPR particles as described herein) or placebo capsule prior to surgery and every 24 hr thereafter (or if discharged, patients are given a supply of MR ondansetron capsules/placebo capsules for self-administration on the appropriate schedule). Patients are asked to rate their symptoms of nausea, as well as any events of nausea or retching over a postoperative period of 48-72 hrs. In addition, the frequency of hospitalization will be recorded. Vomiting is evaluated as a binary event (yes or no), and nausea is quantified on a numeric 11 point scale (0 to 10). The target treatment efficacy is a 50% reduction, i.e., a decrease in incidence from 40% to 20% events. To achieve a 50% reduction, 90 and 120 evaluateable patients are required for 80% and 90% CI (confidence intervals), respectively.

The results of the modeling showed that oral administration of once-daily MR ondansetron capsules is as effective, if not superior to Zofran administered bid in preventing nausea and/or vomiting.

We claim:

1. A method of treating or preventing PONV or PDNV comprising orally administering to a surgical patient in need thereof, at least one extended release dosage form comprising a selective serotonin 5-HT₃ antagonist, prior to and/or after surgery.

2. The method of claim 1, wherein the extended release dosage form comprises TPR particles and IR particles;
the TPR particles each comprise a core coated with a TPR layer;
the core comprises a selective serotonin 5-HT₃ antagonist and a pharmaceutically acceptable organic acid, wherein the selective serotonin 5-HT₃ antagonist and the pharmaceutically acceptable organic acid are separated from each other by an SR layer;
the TPR layer comprises a water insoluble polymer and an enteric polymer;
the SR layer comprises a water insoluble polymer; and
the IR particles each comprise the selective serotonin 5-HT₃ antagonist, and release at least about 80 wt. % of the selective serotonin 5-HT₃ antagonist in about 5 minutes when dissolution tested using United States Pharmacopoeia dissolution methodology (Apparatus 2—paddles@ 50 RPM, 0.1N HCl at 37° C.

3. The method of claim 1, wherein the selective serotonin 5-HT₃ antagonist is selected from the group consisting of ondansetron, tropisetron, granisetron, dolasetron, palonosetron, ramosetron, and salts and/or solvates thereof.

4. The method of claim 1, wherein the selective serotonin 5-HT₃ antagonist is ondansetron, and salts and/or solvates thereof.

5. The method of claim 2, wherein:
the water-insoluble polymer of the TPR and SR layers is independently selected from the group consisting of ethyl cellulose, cellulose acetate, polyvinyl acetate, neutral copolymers of ethyl acrylate and methylmethacrylate, copolymers of acrylic and methacrylic esters containing quaternary ammonium groups, and waxes;
the enteric polymer is selected from the group consisting of cellulose acetate phthalate, hydroxypropyl methylcellulose phthalate, hydroxypropyl methylcellulose acetate succinate, polyvinyl acetate phthalate, pH-sensitive copolymers of methacrylic acid and methylmethacrylate, and shellac; and
the pharmaceutically acceptable organic acid is selected from the group consisting of citric acid, lactic acid, fumaric acid, malic acid, maleic acid, tartaric acid, succinic acid, oxalic acid, aspartic acid, and glutamic acid.

6. The method of claim 5, wherein the TPR and/or SR layers each independently further comprise a pharmaceutically acceptable plasticizer.

7. The method of claim 6, wherein the pharmaceutically acceptable plasticizer of the TPR and/or SR layer(s) is independently selected from the group consisting of triacetin, tributyl citrate, triethyl citrate, acetyl tri-n-butyl citrate, diethyl phthalate, dibutyl sebacate, polyethylene glycol, polypropylene glycol, castor oil, acetylated mono- and di-glycerides and mixtures thereof.

8. The method of claim 2, wherein the TPR particles each comprise:
an inert bead;
an acid layer disposed over the inert bead, comprising the pharmaceutically acceptable organic acid;
the SR layer disposed over the acid layer;
a drug layer disposed over the SR layer, wherein the drug layer comprises the selective serotonin 5-HT₃ antagonist; and
the TPR layer disposed over the drug layer.

9. The method of claim 8, wherein the inert bead has an average particle size of 25-30 mesh and is selected from the group consisting of sugar spheres, cellulose spheres, lactose spheres, lactose-MCC spheres, mannitol-MCC spheres, and silicone dioxide spheres.

10. The method of claim 9, wherein:
the acid layer comprises fumaric acid;
the SR layer comprises ethylcellulose;
the drug layer comprises ondansetron or a pharmaceutically acceptable salt or solvate thereof; and
the TPR layer comprises ethyl cellulose and hydroxypropylmethylcellulose phthalate.

11. The method of claim 2, wherein the IR particles further comprise a pharmaceutically acceptable organic acid, and the pharmaceutically acceptable organic acid of the IR and TPR particles are the same or different.

12. The method of claim 11, wherein the IR particles are a granulate comprising the pharmaceutically acceptable organic acid, the selective serotonin 5-HT₃ antagonist, and an optional binder.

13. The method of claim 12, wherein the IR particles are a granulate comprising fumaric acid, ondansetron or pharmaceutically acceptable salts and/or solvates thereof, and a binder.

14. The method of claim 10, wherein the IR particles are a granulate comprising fumaric acid, ondansetron or pharmaceutically acceptable salts and/or solvates thereof, and a binder.

15. The method of claim 14, wherein the extended release dosage form is a capsule comprising a therapeutically effective amount of the TPR particles and IR particles, whereby the capsule contains a total amount of ondansetron or pharmaceutically acceptable salts and/or solvates thereof equivalent to 24 mg of ondansetron.

16. The method of claim 1, wherein said administering comprises administering the extended release dosage form prior to surgery, whereby PONV and/or PDNV are ameliorated or prevented.

17. The method of claim 15, wherein said administering comprises administering the extended release dosage form prior to surgery, whereby PONV and/or PDNV are ameliorated or prevented.

18. The method of claim 1, wherein said administering comprises administering the extended release dosage form after surgery and at discharge, whereby PDNV is ameliorated or prevented.

19. The method of claim 15, wherein said administering comprises administering the extended release dosage form after surgery and at discharge, whereby PDNV is ameliorated or prevented.

20. The method of claim 1, wherein said administering comprises administering the extended release dosage form after discharge, whereby PDNV is ameliorated or prevented.

21. The method of claim 15, wherein said administering comprises administering the extended release dosage form after discharge, whereby PDNV is ameliorated or prevented.

22. The method of claim 18, further comprising administering at least one additional extended release dosage form once daily after discharge.

23. The method of claim 19, further comprising administering at least one additional extended release dosage form once daily after discharge.

24. The method of claim 18, further comprising administering at least one additional extended release dosage form in the morning following discharge, and optionally once-daily for up to 4 additional days.

25. The method of claim 19, further comprising administering at least one additional extended release dosage form in the morning following discharge, and optionally once-daily for up to 4 additional days.

26. The method of claim 1, further comprising administering an intravenous antiemetic before or immediately after surgery.

27. The method of claim 1, wherein the patient is at moderate or increased risk of PONV or PDNV.

28. The method of claim 16, wherein the patient is at moderate or increased risk of PONV or PDNV

29. The method of claim 27, and wherein the patient meets one or more of the following criteria:
(a) female;
(b) prior history of PONV and/or motion sickness;
(c) nonsmoker
(d) the patient has had outpatient surgery;
(e) the patient has had outpatient surgery having a duration of at least 60 min.;
(f) the patient has had general anesthesia which is balanced inhalational anesthesia.
(g) the patient has had nitrous oxide anesthesia;
(h) the patient has been administered intraoperative or post operative opioids; and
(i) the patient has had a surgery selected from the group consisting of laparoscopy, ear-nose-throat, neurosurgery, breast surgery, strabismus surgery, laparotomy, and plastic surgery.

30. The method of claim 1, wherein the surgical patient is treated with opioid analgesics after surgery.

31. The method of claim 30, wherein the opioid is selected from the group consisting of codeine, morphine, thebaine, oripavine, diacetyl morphine, dihydrocodeine, hydrocodone, hydromorphone, nicomorphine, oxycodone, oxymorphone, fentanyl, α-methyl fentanyl, alfentanil, sufentanil, remifentanil, meperidine, buprenorphine, etorphine, methadone, and tramadol.

32. The method of claim 15, wherein the surgical patient is treated with opioid analgesics after surgery.

33. The method of claim 32, wherein the opioid is selected from the group consisting of codeine, morphine, thebaine, oripavine, diacetyl morphine, dihydrocodeine, hydrocodone, hydromorphone, nicomorphine, oxycodone, oxymorphone, fentanyl, α-methyl fentanyl, alfentanil, sufentanil, remifentanil, meperidine, buprenorphine, etorphine, methadone, and tramadol.

34. The method of claim 1, further comprising administering at least one additional oral antiemetic.

35. The method of claim 34, wherein the additional oral antiemetic is selected from the group consisting of NK-1 antagonists, dopamine antagonists, H1 histamine receptor antagonists, cannabinoids, benzodiazepines, anticholinergics, and steroids.

36. The method of claim 35, wherein the NK-1 antagonist is selected from the group consisting of aprepitant and casopitant; the dopamine antagonist is selected from the group consisting of domperidone, droperidol, haloperidol, chlorpromazine, and prochlorperazine; the H1 histamine receptor antagonist is selected from the group consisting of cyclizine, diphenhydramine, dimenhydrinate, meclizine, promethazine, and hydroxyzine; the cannabinoid is selected from the group consisting of cannabis, dronabinol, and nabilone; the benzodiazepine is selected from the group consisting of midazolam and lorazepam; the anticholinergic is scopalamine; and the steroid is dexamethasone.

37. The method of claim 15, further comprising administering at least one additional oral antiemetic.

38. The method of claim 37, wherein the additional oral antiemetic is selected from the group consisting of NK-1 antagonists, dopamine antagonists, H1 histamine receptor antagonists, cannabinoids, benzodiazepines, anticholinergics, and steroids.

39. The method of claim 38, wherein the NK-1 antagonist is selected from the group consisting of aprepitant and casopitant; the dopamine antagonist is selected from the group consisting of domperidone, droperidol, haloperidol, chlorpromazine, and prochlorperazine; the H1 histamine receptor antagonist is selected from the group consisting of cyclizine, diphenhydramine, dimenhydrinate, meclizine, promethazine, and hydroxyzine; the cannabinoid is selected from the group consisting of cannabis, dronabinol, and nabilone; the benzodiazepine is selected from the group consisting of midazolam and lorazepam; the anticholinergic is scopalamine; and the steroid is dexamethasone.
